(19) United States
(12) Patent Application Publication
(10) Pub. No.: US 2016/0152540 A1
(43) Pub. Date: Jun. 2, 2016

(54) NEW PROCESS FOR THE MAINTAINING OF A RATIO OF ISOMERS OF CAROTENOID COMPOUNDS

(71) Applicant: DSM IP ASSETS B.V., The Heerlen (NL)

(72) Inventors: Basel ULRICH, Basel (CH); Christian SCHAEFER, Basel (CH); Franziska KREISSIG, Basel (CH)

(21) Appl. No.: 14/900,331

(22) PCT Filed: Jun. 18, 2014
(86) PCT No.: PCT/EP2014/062842

§ 371 Date: Dec. 21, 2015

(30) Foreign Application Priority Data
Jun. 21, 2013 (EP) 13173229.9

(51) Int. Cl. C07C 45/86; C07B 63/04

(52) U.S. Cl. CPC C07C 45/86; A23L 1/30; A23K 1/1609; C07B 63/04; A23V 2002/00; C07B 2200/09; C07C 2101/16

(57) ABSTRACT

The present invention relates to process for the maintaining of a ratio stereoisomers of carotenoid compounds by using at least one alkali and/or earth alkali metal salt of an organic acid, to specific formulations and to the use of such specific formulations.

The present invention relates to a process for the maintaining of a ratio of stereoisomers of carotenoid compounds, to specific formulations and to the use of such specific formulations.

Carotenoids (which are also named carotenoid compounds in the context of the present patent application) generally consist of conjoined units of the hydrocarbon isoprene, with alternating single and double bonds. The carotenoids absorb light energy of certain frequencies and transfer it to chlorophyll for use in photosynthesis.

Carotenoids are nutritionally important for animals as well as human beings, and also have antioxidant properties.

The carotenoids are classified into two groups:
carotenes (do only contain H and C atoms), and
xanthophylls (do also contain O-atoms).

The carotenoids, when found in nature, are mainly existing in the all-trans stereoisomeric form. But the carotenoids have a tendency to steroisomerise (that means to transform into other stereoisomeric forms). In the context of the present invention E-forms, Z-forms and mixtures of E/Z-forms are meant by stereoisomeric forms. Stereoisomerisation usually results in a mixture of many different stereoisomeric structures: such as:

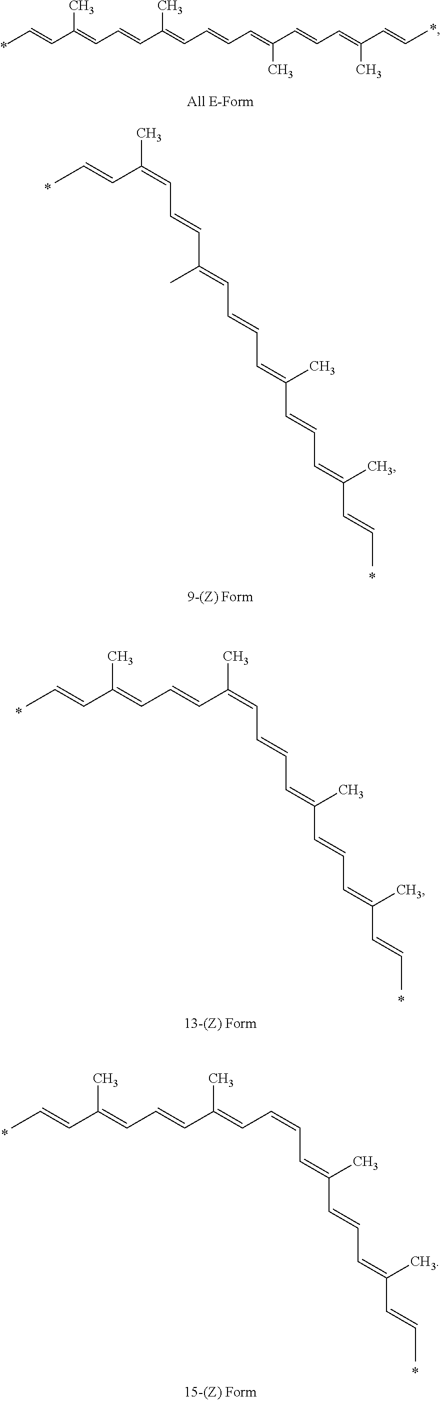

The “*”'s (=asterisks) are the positions of the endgroups (R₁ and R₂)

All stereoisomeric forms of the carotenoid compounds usually have different proper- ties in regard to i.e. light absorption, bioavailability etc. So when the isomerisation takes place the properties of a formulation comprising carotenoid compounds can change.

Therefore the goal of the present invention was to find a process, which maintains a ratio of stereoisomers of carotenoid compounds, so that the properties of a formulation comprising carotenoids are not changing.

From the prior art (WO2013010820) it is known that specific mineral salts are able to stabilize a ratio of stereoisomers of carotenoid compounds.

Due to the importance of such a process there is a constant need for improvement.

Surprisingly, it was found that the isomerisation of carotenoid compounds is influenced by the addition of at least one alkali and/or earth alkali metal salt of an organic acid. This addition results that the stereoisomeric form (or also a mixture of two or more stereoisomeric forms) are stabilised. The stereoisomerisation is prevented or slowed down.

Therefore, the present invention relates to a process for the maintaining of a ratio of stereoisomers of carotenoid compounds of formula (I)

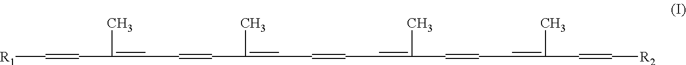

wherein R₁ and R₂ are independently of each other

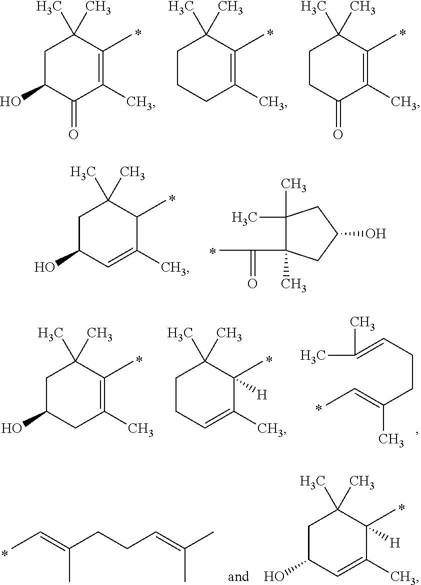

characterised in that at least one compound of formula (I)

is mixed with less than 1 weight-% (wt-%), based on the total weight of the compound(s) of formula (I), of at least one alkali and/or earth alkali metal salt of an organic acid.

The asterisks mark the bond to the backbone. Usually the carotenoid compounds are present in the form of a solution or emulsion. The solution or emulsion can then in a further step be dried (spray dried, freeze dried), mixed with other formulations or ingredients, etc.

The concentration of the carotenoid compound in such a formulation can vary depending on the use of such a formulation. Suitable solvents are such wherein carotenoid compounds are soluble, such as ethylacetate, methylenechloride, chloroform, acetone, etc.

If an emulsion system is used, then it is usually an oil-in-water emulsion. For such an emulsion any commonly known and used oils and emulsifiers are used. The choice of the oil as well as the emulsifier depends on the use of the formulation. In case a food or feed product is produced, then these compounds need to be food or feed grade.

Suitable oils can be from any origin. They can be natural, modified or synthetic. If the oils are natural, they can be plant or animal oils. Suitable oils are i.e. coconut oil, corn oil, cottonseed oil, olive oil, palm oil, peanut oil, canola oil, safflower oil, sesame oil, soybean oil, sunflower oil, hazelnut oil, almond oil, cashew oil, macadamia oil, mongongo nut oil, pracaxi oil, pecan oil, pine nut oil, pistachio oil, sacha Inchi (Plukenetia volubilis) oil, walnut oil, polyunsaturated fatty acids (such as triglyceride and/or ethyl ester, (for example arachidonic acid, eicosapentaenoic acid, docosahexaenoic acid and γ-linolenic acid and/or ethyl ester).

Any commonly known and used emulsifier can be used. The emulsifier can be chosen depending on the final use of the formulation afterwards. Suitable emulsifiers are i.e. modified (food) starches, pectin, alginate, carrageenan, furcellaran, chitosan, maltodextrin, dextrin derivatives, celluloses and cellulose derivatives (e.g. cellulose acetate, methyl cellulose, hydroxypropyl methyl cellulose), lignosulfonate, polysaccharide gums (such as gum acacia, gum arabic, flaxseed gum, ghatti gum, tamarind gum and arabinogalactan), gelatine (bovine, fish, pork, poultry), plant proteins (such as concentrates, isolates, hydrolysates, etc. from peas, soybeans, castor beans, cottonseed, potatoes, sweet potatoes, manioc, canola, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, PG ester and sugar ester (as well as derivatives thereof).

The starches can be modified physically and chemically. Pregelatinized starches are examples of physically modified starches.

Acidic modified, oxidized, OSA-modified, cross-linked, starch esters, starch ethers and cationic starches are examples of chemically modified starches.

The formulations can comprise further auxiliary agents. Depending for which use the formulations are used, the auxiliary agent(s) can vary. These auxiliary agents can be useful for the formulation by further improving its properties, such as physical stability, storage stability, visual perception, etc. Auxiliaries can also be useful for the application in the food, feed or personal care product by improving the property of these compositions, physical stability, storage stability, visual perception, controlled release in the GI-tract, pH control, oxidation resistant, etc. The concentration of these auxiliaries can vary, depending on the use of these auxiliaries. Preferred carotenoid compounds are the following ones:

Compound of Formula (Ia):

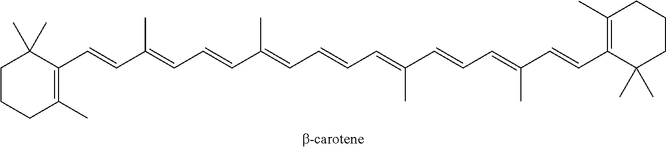 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

Compound of Formula (Ib):

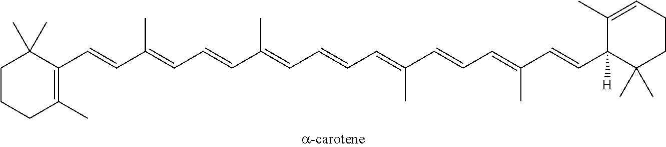 [SMILES: CC1=CCCC(C)(C)[C@H]1/C=C/C(C)=C/C=C/C(C)=C/C=C/C=C(C)/C=C/C=C(C)/C=C/C1=C(C)CCCC1(C)C]

Compound of Formula (Ic):

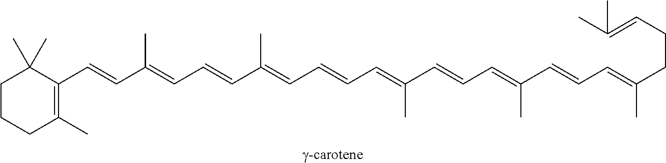 [SMILES: CC(C)=CCC/C(C)=C/C=C/C(C)=C/C=C/C(C)=C/C=C/C=C(C)/C=C/C=C(C)/C=C/C1=C(C)CCCC1(C)C]

Compound of Formula (Id):

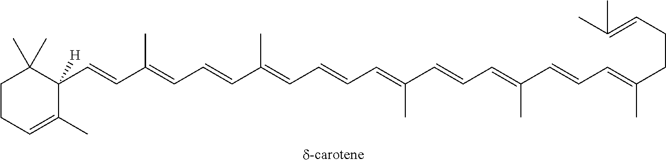 [SMILES: CC(C)=CCC/C(C)=C/C=C/C(C)=C/C=C/C(C)=C/C=C/C=C(C)/C=C/C=C(C)/C=C/[C@H]1C(C)=CCCC1(C)C]

Compound of Formula (Ie):

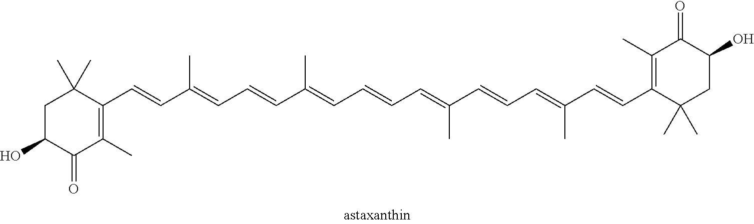 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

Compound of Formula (If):

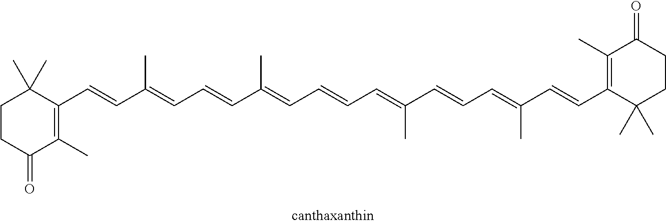 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

Compound of Formula (Ig):

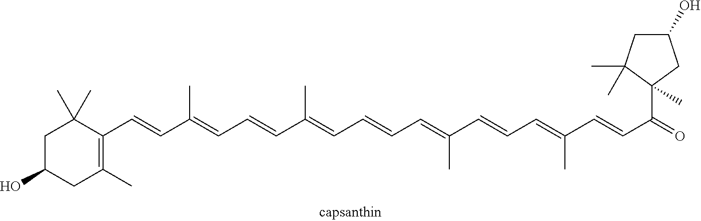 [SMILES: CC1=C(/C=C/C(C)=C/C=C/C(C)=C/C=C/C=C(C)/C=C/C=C(C)/C=C/C(=O)[C@]2(C)C[C@@H](O)CC2(C)C)C(C)(C)C[C@H](O)C1]

Compound of Formula (Ih):

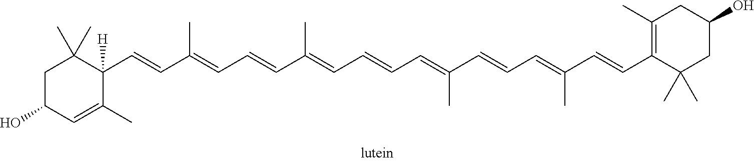 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

Compound of Formula (Ii):

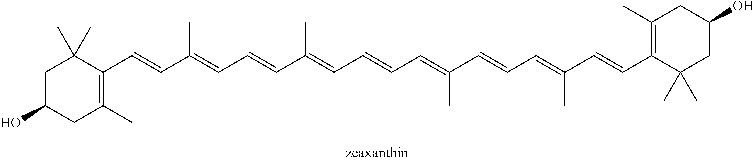 [SMILES: CC1=C(/C=C/C(C)=C/C=C/C(C)=C/C=C/C=C(C)/C=C/C=C(C)/C=C/C2=C(C)C[C@@H](O)CC2(C)C)C(C)(C)C[C@H](O)C1]

Compound of Formula (Ij):

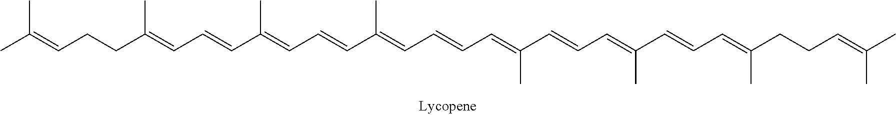 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

To stabilise a ratio of the carotenoid compounds at least one alkali and/or earth alkali metal salt of an organic acid is added in an amount of less than 1 wt-%, based on the total weight of the carotenoid compound or mixture thereof. Preferably less than 0.9 wt-%, more preferably less than 0.8 wt-%. Usually an amount of 0.0001 wt-% to 1wt-%, based on the total weight of the carotenoid compound or mixture thereof, of at least one alkali and/or earth alkali metal of an organic acid is used.

The process is usually carried out at a temperature from 15° C. to 160° C., preferably 20° C. to 130° C., more preferably 20° C. to 110° C.

The process is usually carried out at a pressure of 1 bar to 10 bar, preferably 1 bar to 8 bar, more preferably 1 bar to 5 bar.

Afterwards the carotenoid compounds comprising at least one alkali and/or earth alkali metal salt of an organic acid in an amount of less than 1 wt-%, can be used as such or be used to be further formulated.

The ratio of the isomers is stabilised in the composition as such (only carotenoid compound and alkali and/or earth alkali metal salts of an organic acid) as well in formulations comprising such a composition.

The organic acid is preferably R-COON, wherein R is a C₁-C₄-alkyl group.

Preferred alkali and earth alkali metals of the alkali and/or earth alkali metal salts of an organic acid are K, Ca and Mg.

Preferred alkali and/or earth alkali metal salts of an organic acid have the following formulae

R-COOMe¹ or (R—COO)₂Me₂,

wherein R is a C₁-C₄-alkyl group, and

Me¹ is K⁺, and

Me² is Ca²⁺or Mg²+.

The most preferred salt of an organic acid is potassium acetate (═CH₃COOK).

In case of β-carotene (compound (Ia)), the stereoisomerisation leads to a shift of the shade of the colour. The All Z-form is more reddish whereas the Z forms are slightly yellowish.

Therefore a preferred embodiment of the present invention relates to a process for maintaining a ratio of stereoisomers of compound of formula (Ia)

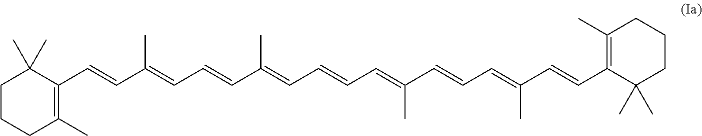 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

characterised in that less than 1 wt-%, based on the total weight of the compound of formula (Ia), of CH₃COOK is added.

In case of astaxanthin(compound (Ie)), the stereoisomerisation leads to different bioavailability of the compound. The all (E) form of astaxanthin has the best bioavailability.

Therefore this form (or a form with a high amount of all (E) at least 85% of all (E)) should be stabilised.

Therefore a preferred embodiment of the present invention relates to a process for maintaining a ratio of stereoisomers of compound of formula (Ie)

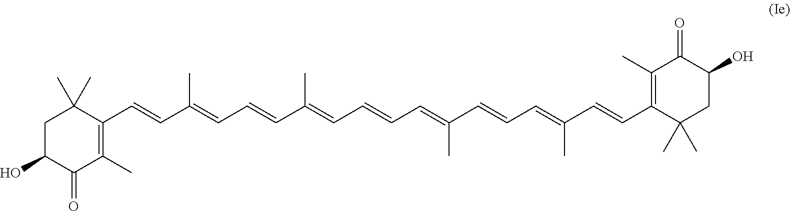 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

characterised in that less than 1 wt-%, based on the total weight of the compound of formula (Ie), of CH₃COOK is added.

The alkali and/or earth alkali salt of an organic acid can be added as a solid form as well as a solution (or a combination of both).

Furthermore the present invention also relates to a formulation comprising at least one carotenoid compound of formula (I)

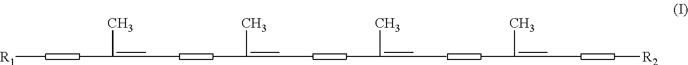

wherein R₁ and R₂ are independently of each other

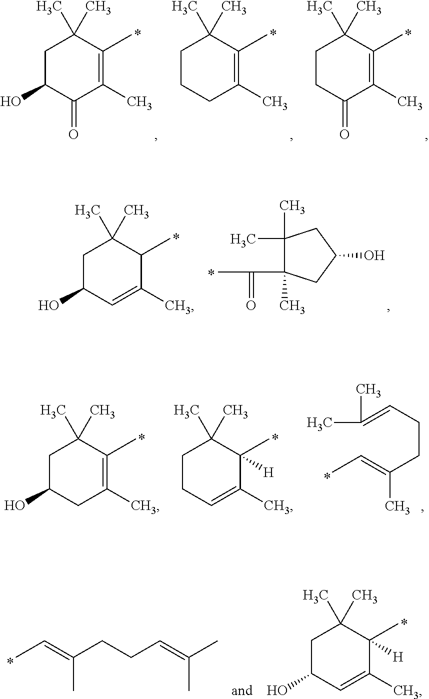

and 0.0001 to 1wt-%, based on the total weight of the compound(s) of formula (I), of at least one alkali and/or earth alkali metal salt of an organic acid.

All preferences for the process also apply for the formulations.

As said above the formulations the carotenoid compounds comprising at least one alkali and/or earth alkali metal salt of an organic acid in an amount of less than 1 wt-%, can be used as such or used to be further formulated.

The formulation as described above can be used in food, feed or personal care products.

The amount of a formulation as described above used in food, feed or personal care products depends on the food, feed or personal care products

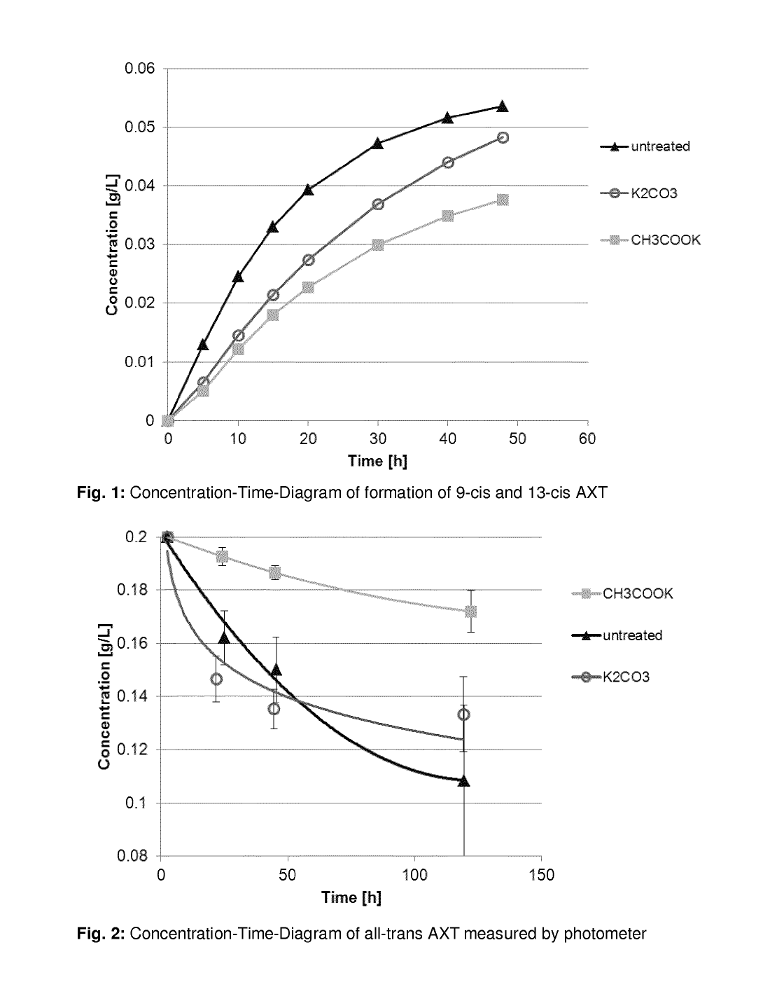

FIG. 1: Concentration-Time-Diagram of formation of 9-cis and 13-cis AXT

FIG. 2.: Concentration-Time-Diagram of all-trans AXT measured by photometer

The following examples serve to illustrate the invention. All percentages are given in relation to weight and the temperature is given in degree Celsius.

### EXAMPLE 1

14.1 mg astaxanthin was dissolved in 70 ml chloroform. 13.7 μg CH₃COOK was added to this solution (by adding an aliquot of a diluted solution comprising 13.7 mg CH₃COOK in 70 ml chloroform). The solution was homogenized and the formulation was tempered at 40° C. The solution was stored and samples were taken periodically and the concentration of astaxanthin was measured spectrophotometrically at 478 nm.

### EXAMPLE 2 (Comparison)

16.1 mg astaxanthin was dissolved in 80 ml chloroform. 16.2 μg K₂CO₃ was added to this solution (by adding an aliquot of a diluted solution comprising 16.2 mg K₂CO₃ in 70 ml chloroform). The solution was homogenized and the formulation was tempered at 40° C. The solution was stored and samples were taken periodically and the concentration of astaxanthin was measured spectrophotometrically at 478 nm.

### EXAMPLE 3 (Comparison)

16.1 mg astaxanthin was dissolved in 80 ml chloroform. The solution was homogenized and the formulation was tempered at 40° C. The solution was stored and samples were taken periodically and the concentration of astaxanthin was measured spectrophotometrically at 478 nm.

The FIG. 1 and the FIG. 2 show that the addition of CH₃COOK, stabilises the all-E-form better than K₂CO₃. The untreated astaxanthin (no addition of any salt) shows how the ratio is deteriorating usually.

1. A process for the maintaining of a ratio of stereoisomers of carotenoid compounds of formula (I) 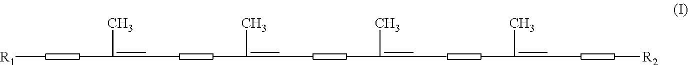
wherein R₁ and R2 are independently of each other 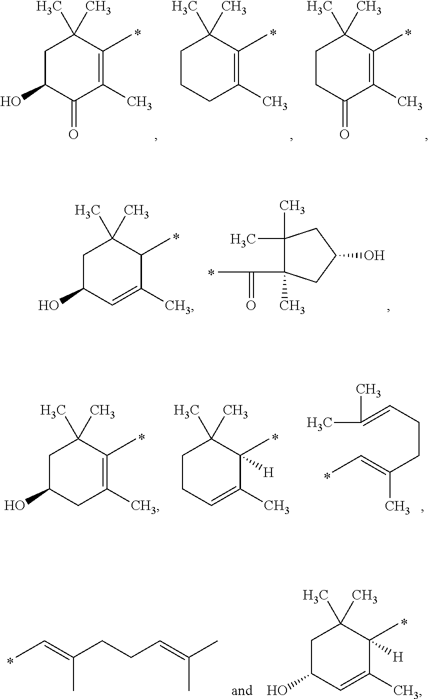
wherein the compounds of formula (I)
are mixed with less that 1 wt-%, based on the total weight of the compound(s) of formula (I), of at least one alkali and/or earth alkali metal salt of an organic acid.

2. Process according to claim 1 wherein the carotenoid compounds are chosen from

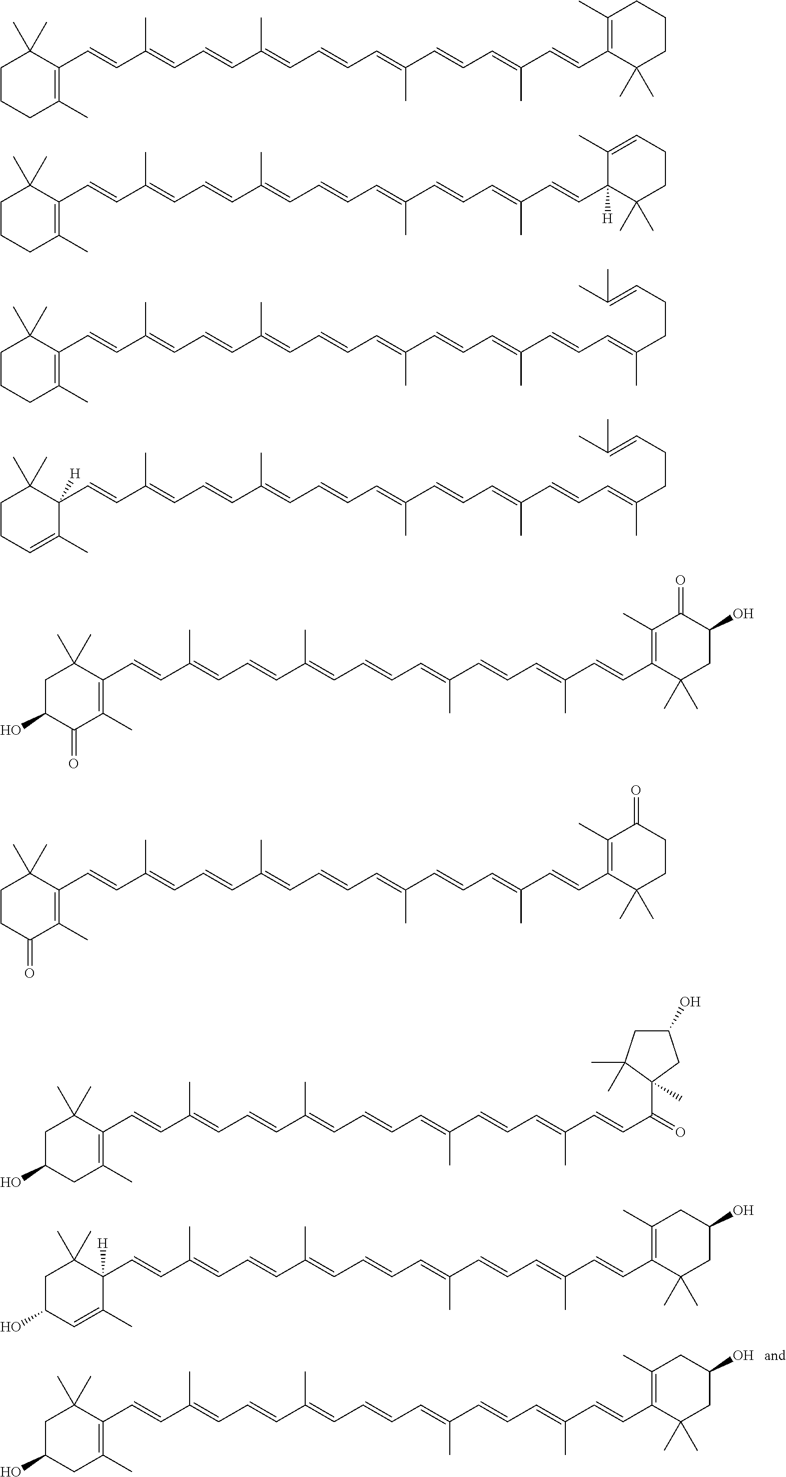

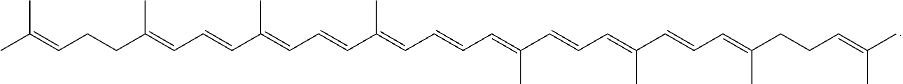 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

3. Process according to claim 1, wherein the carotenoid compound is

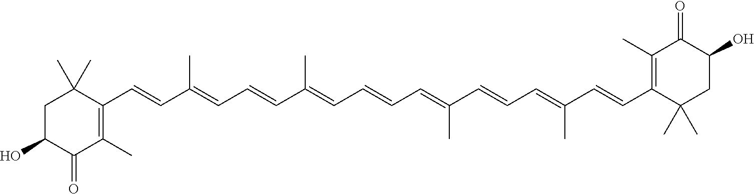 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

4. Process according to claim 1, wherein the carotenoid compound is

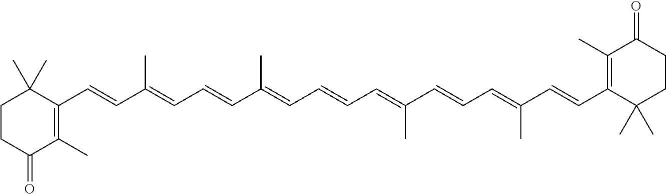 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

5. Process according to claim 1 wherein the alkali and/or earth alkali metal salts of an organic acid are compounds of formulae
R—COOMe¹ or (R—COO)₂Me₂,
wherein R is a C₁-C4-alkyl group, and
Me¹ is an alkali cation, and
Me² is an earth alkali cation.

6. Process according to claim 1, wherein the alkali and/or earth alkali metal cations are K⁺, Ca²+and/or Mg²+ salts.

7. Process according to claim 1, wherein CH₃COOK used.

8. Process according to claim 1 wherein 0.0001 wt-% to 1 wt. %, based on the total weight of the compound(s) of formula (I), of the alkali and/or earth alkali metal salt of an organic acid is used.

9. A formulation comprising at least one carotenoid compound of formula (I) 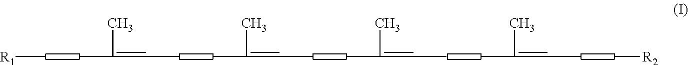
wherein R₁ and R₂ are independently of each other 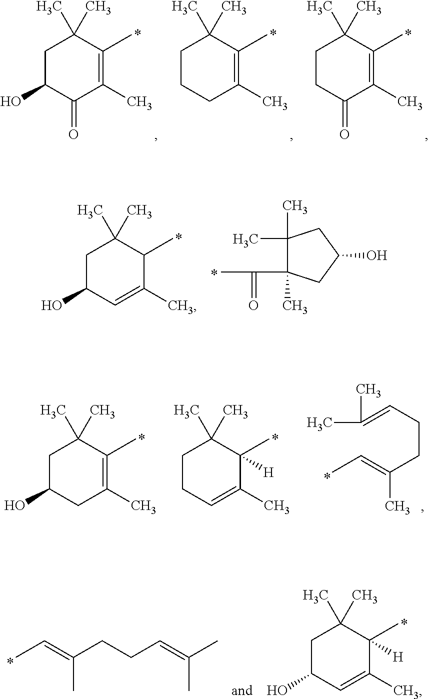
and 0.0001 to 1 wt-%, based on the total weight of the compound(s) of formula (I), of at least one alkali and/or earth alkali metal salt of an organic acid.

10. Formulation according to claim 9 wherein the carotenoid compounds are chosen from

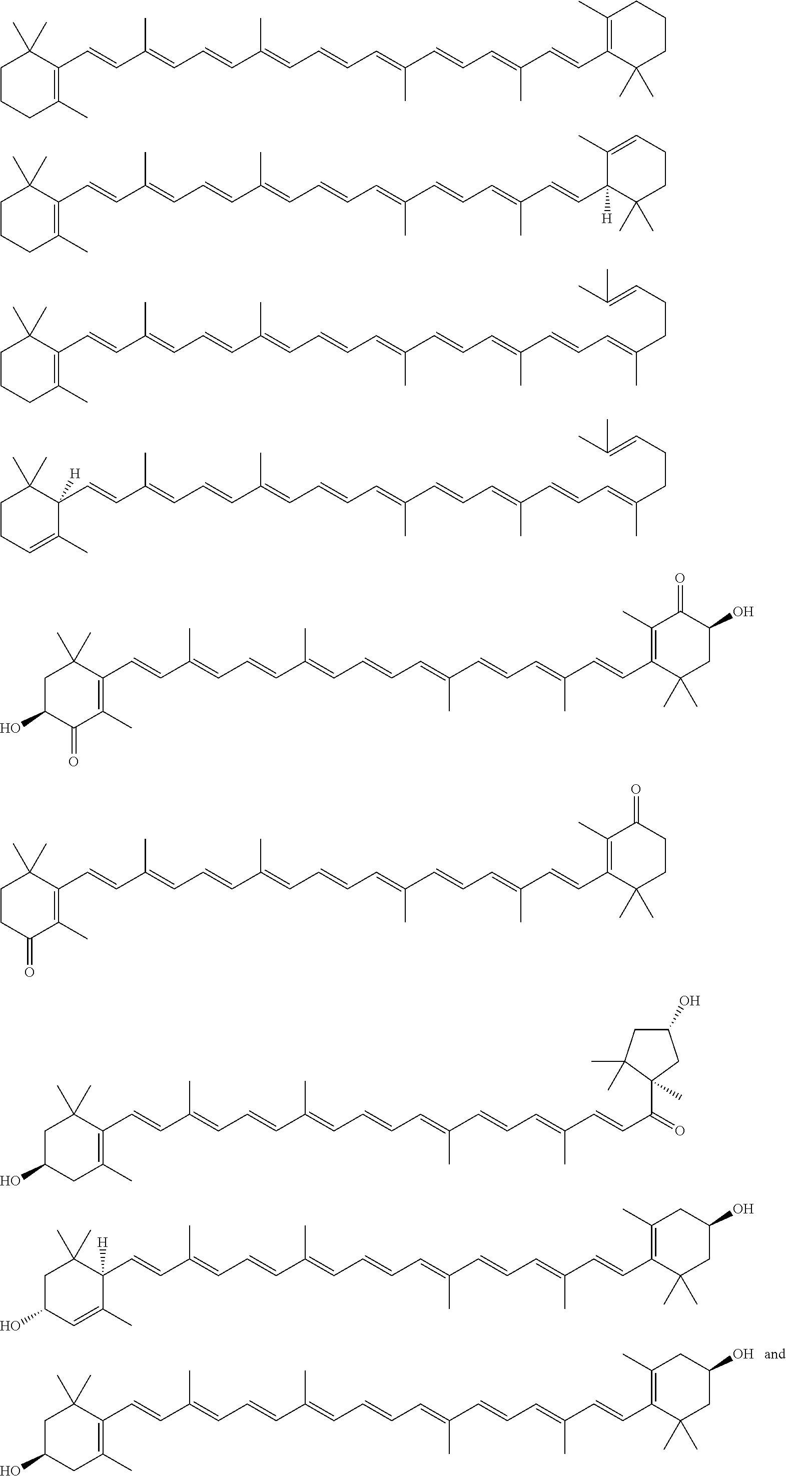

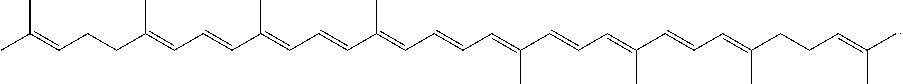 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

11. Formulation according to claim 9, wherein the carotenoid compound is

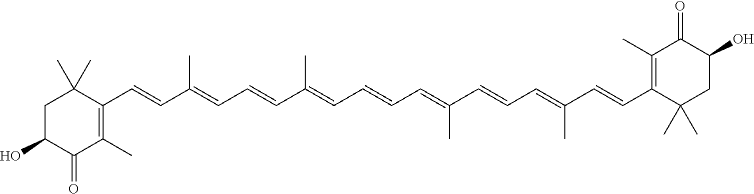 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

12. Formulation according to claim 9, wherein the carotenoid compound is

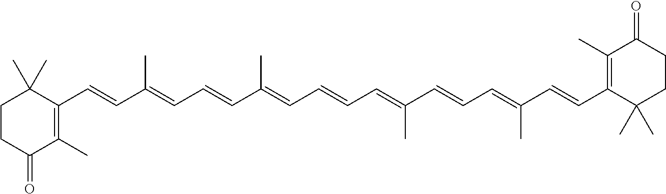 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

13. Formulation according to claim 9 wherein the alkali and/or earth alkali metal salts are salts from an organic acid of formulae
R—COOMe¹ or (R—COO)₂Me₂,
wherein R is a C₁-C4-alkyl group, and
Me¹ is an alkali cation, and
Me² is an earth alkali cation.

14. Formulation according to claim 9, wherein the alkali and/or earth alkali metal cations of the alkali and/or earth alkali metal of the organic acids are K⁺, Ca²+ and/or Mg²⁺.

15. Formulation according to claim 9, wherein CH₃COOK is the salt.

16. Formulation according to claim 1 wherein 0.0001 wt-% to 1 wt-%, based on the total weight of the compound(s) of formula (I), of the alkali and/or earth alkali metal salt of an organic acid is comprised.

17. Use of at least one formulation according to claim 9 in food, feed or personal care products.

18. Food, feed or personal care product comprising at least one formulation of claim 9.
